(19) United States
(12) Patent Application Publication
(10) Pub. No.: US 2015/0045244 A1
(43) Pub. Date: Feb. 12, 2015

(54) METHOD FOR DETERMINING RHEUMATOID ARTHRITIS ACTIVITY INDICATOR, AND BIOMARKER USED THEREIN

(71) Applicants: Tsutomu Takeuchi, Tokyo (JP); Koichi Amano, Saitama (JP); Yohei Ishizawa, Kanagawa (JP); Seiji Nakamura, Kanagawa (JP); Motohiko Tanino, Kanagawa (JP); Yuko Hata, Kanagawa (JP)

(72) Inventors: Tsutomu Takeuchi, Tokyo (JP); Koichi Amano, Saitama (JP); Yohei Ishizawa, Kanagawa (JP); Seiji Nakamura, Kanagawa (JP); Motohiko Tanino, Kanagawa (JP); Yuko Hata, Kanagawa (JP)

(73) Assignees: DNA CHIP RESEARCH INC., Kanagawa (JP); SAITAMA MEDICAL UNIVERSITY, Saitama (JP); KEIO UNIVERSITY, Tokyo (JP)

(21) Appl. No.: 14/358,517

(22) PCT Filed: Nov. 17, 2011
(86) PCT No.: PCT/JP2011/076584

§ 371 Date: Oct. 1, 2014

(51) Int. Cl. C12Q 1/68

(52) U.S. Cl. CPC C12Q 1/6883; C12Q 2600/158; C12Q 2600/112
USPC 506/9; 536/23.1; 506/16

(57) ABSTRACT

The object of the invention is to find a simple biomarker with high reproducibility with regard to a method for evaluating the activity of the condition and progression of rheumatoid arthritis disease and provide an effective evaluation method therefor. The invention is to provide a method for determining the value of a rheumatoid arthritis-linked indicator in a subject, the method being characterized by comprising a step for measuring the amount of expression of the FAM20A gene in blood collected from the subject, a step for analyzing the measured amount of expression using a gene expression profile that is prepared in advance and correlates with the indicator and a step for estimating the value of the indicator in the subject on the basis of the analysis results.

### FIELD OF THE INVENTION

The present invention relates to a method for determining a value of a rheumatoid arthritis-linked indicator in a subject and a biomarker used therein. More specifically, the invention relates to a method for measuring and normalizing an amount of expression of a biomarker gene according to the present invention in blood collected from a subject and then determining a rheumatoid arthritis activity indicator in the subject on the basis of a gene expression profile prepared from the amount of expression.

### BACKGROUND OF THE INVENTION

Rheumatoid Arthritis (RA), a typical rheumatic disease, is a systemic connective tissue disorder having chronic polyarthritis as the major symptom and is one type of autoimmune diseases. Rheumatoid arthritis frequently occurs among women of 30 to 60 years old, and it is estimated that the number of patients suffering from this disease is about 700,000 in Japan. The condition of rheumatoid arthritis includes angiogenesis in the arthrosynovial membrane, inflammatory cell infiltration, synoviocyte proliferation, and cartilage and bone destruction. In rheumatoid arthritis, arthritis progresses while repeating amelioration and exacerbation and gradually destroys cartilages and bones, thereby inhibiting daily activities of patients with rheumatoid arthritis and lowering their quality of life (QOL).

In the clinical rheumatoid arthritis, the activity of the condition and progression of rheumatoid arthritis in patients has been evaluated by calculating a specific indicator in order to provide the best therapy to those patients. By way of example, in evaluating this indicator, the activity of the condition and progression of rheumatoid arthritis has been scored by combining various evaluation items including the self-evaluation of pain by a patient, the examination and measurement of joints (pain joint count, tender joint count, swollen joint count, etc.) and blood examination (erythrocyte sedimentation rate, rheumatoid factor, CRP, etc.). Based on the scored indicator, the degree of disorder of the patient is evaluated. It is also reported that an urinary interleukin-1 receptor antagonist can be used as an indicator (Japanese Patent No. 3530239).

### SUMMARY OF THE INVENTION

In the case of evaluating the activity of the condition and progression of rheumatoid arthritis using the abovementioned indicator, patients' self-evaluation of pains is necessarily involved as described above, and therefore patients' subjective judgment is required. However, since patients' own evaluation of pains and the like fluctuates depending on individuals and observers, it is difficult to make objective evaluation of the activity. Moreover, it is also difficult to comprehensively evaluate the condition and progression of patients using currently-used markers in blood alone. Accordingly, in order to diagnose rheumatoid arthritis disease and estimate a prognosis after treatment, there is a need to find a simple marker for the disease with high reproducibility and develop a method for directly determining the degree of the disease.

The present invention was made under the abovementioned circumstances, and the purpose of the invention is to find a simple biomarker with high reproducibility with regard to a method for evaluating the activity of the condition and progression of rheumatoid arthritis disease and to provide an effective evaluation method therefor.

In order to solve the abovementioned problems, the present inventors paid attention to the amount of expression of the FAM20A gene originated from human patients with rheumatoid arthritis disease and thereby found that the amount of expression of the FAM20A gene changes in proportion to the level of the activity of the condition of rheumatoid arthritis disease. Accordingly, as a result of intensive studies on the amount of expression of the FAM20A gene, they found that FAM20A in blood collected from a subject could be a diagnostic marker for rheumatoid arthritis disease as well as a marker for the effect of administering therapeutic drugs and that the activity of the condition and progression of rheumatoid arthritis disease could be evaluated by determining the amount of expression of the FAM20A gene.

More specifically, according to a first main point of the present invention, provided is a method for determining a value of a rheumatoid arthritis-linked indicator in a subject, comprising measuring an amount of expression of the FAM20A gene in blood derived from the subject, analyzing the measured amount of expression using a preliminarily-prepared gene expression profile correlating with the indicator, and estimating the value of the indicator in the subject on the basis of the analysis results.

In this constitution, the value of a rheumatoid arthritis-linked indicator can be determined in a subject simply by taking a blood sample without placing a special burden on the subject. Moreover, in this constitution, the value of a rheumatoid arthritis-linked indicator can be determined in a low invasive and simple manner.

Since the value of a rheumatoid arthritis-linked indicator can be determined by measuring the amount of expression of the FAM20A gene in blood, reproducibility is high, reliability is also high as compared with the conventional method including patients' self-evaluation, and examination can be made with ease. Moreover, the use of the method according to the present invention enables to determine the therapeutic effect of a therapeutic drug for rheumatoid arthritis. This method is useful because subjective judgment of pains and the like by patients is not required such that the effect can be determined objectively.

Furthermore, according to one embodiment of the present invention, in the abovementioned method, the indicator is preferably selected from the group consisting of DAS28-CRP, DAS28-ESR, DAS44, EULAR improvement criteria, CDAI, SDAI, ACR and serum CRP concentration. In this case, the indicator shows the progression of rheumatoid arthritis or enables to determine a therapeutic effect of therapeutic drugs for rheumatoid arthritis.

Furthermore, according to another embodiment of the present invention, in the abovementioned method, the analyzing is preferably performed by comparing the measured amount of expression of the FAM20A gene with a gene expression profile that is prepared based on an amount of expression of the FAM20A gene in blood collected from a patient with rheumatoid arthritis and shows a correlation between an amount of expression of the FAM20A gene and the indicator. In this case, the gene expression profile shows a positive correlation, and wherein the indicator shows high activity of progression when the amount of expression of the FAM20A gene is large.

Furthermore, according to another embodiment of the present invention, in the abovementioned method, the FAM20A gene preferably consists of (i) a base sequence according to SEQ ID NO: 1 or (ii) a base sequence that hybridizes with the base sequence according to SEQ ID NO: 1 under stringent conditions, the base sequence being a base sequence encoding a protein having a function equivalent to that of a protein consisting of an amino acid sequence according to SEQ ID NO: 2.

Furthermore, according to another embodiment of the present invention, in the abovementioned method, the subject is preferably suffering from rheumatoid arthritis. In this case, the measuring measures amounts of expression of the FAM20A gene derived from blood before and after administering a therapeutic agent for rheumatoid arthritis to the subject, wherein a therapeutic effect of the therapeutic agent for rheumatoid arthritis is determined by analyzing the amounts of expression of the FAM20A gene in blood before and after administration using the gene expression profile.

Furthermore, according to a second main point of the present invention, provided is a FAM20A biomarker for determining a value of a rheumatoid arthritis-linked indicator in a subject, comprising a base sequence according to SEQ ID NO: 1.

Furthermore, according to a third main point of the present invention, provided is the use of FAM20A comprising a base sequence according to SEQ ID NO: 1 as a biomarker for determining a value of a rheumatoid arthritis-linked indicator in a subject.

Furthermore, according to a fourth main point of the present invention, provided is an array used in the abovementioned method, wherein a probe that hybridizes with at least part of a base sequence encoding the FAM20A gene under stringent conditions is immobilized at a position on a solid support.

The features and marked operation and effects of the present invention other than those described above will be obvious to those skilled in the art by referring to detailed description of the invention and drawings as shown below.

### BRIEF DESCRIPTION OF THE DRAWINGS

FIG. 1 is a graph showing the analysis of cells expressing the FAM20A gene according to the present invention.

FIG. 2 is a scatter plot graph showing the analysis results of the correlation between the amount of expression of the FAM20A gene according to the present invention and DAS28-CRP as well as DAS28-ESR.

FIG. 3A shows changes in the amount of expression of the FAM20A gene according to the present invention before and after administering methotrexate.

FIG. 3B shows changes in the amount of expression of the FAM20A gene according to the present invention before and after administering infliximab.

FIG. 4 is a graph showing the analysis of expression of the FAM20A gene according to the present invention in normal individuals and patients with inflammatory diseases.

FIG. 5A is a graph showing the correlation between microarrays of the FAM20A gene according to the present invention and PCR as a result of analyzing expression.

FIG. 5B is a graph showing the significant difference between a normal individual and a patient with rheumatoid arthritis with regard to the amount of expression of the FAM20A gene according to the present invention.

FIG. 5C is a graph showing the correlation between DAS28-CRP, which is an indicator for the activity of disease, and CRP, which is clinical information, with regard to the amount of expression of the FAM20A gene according to the present invention.

### DETAILED DESCRIPTION OF THE INVENTION

In the following, one embodiment of the present invention and examples are given with reference to drawings.

As described above, the method according to the present embodiment for determining the value of a rheumatoid arthritis-linked indicator in a subject is characterized by comprising a step for measuring the amount of expression of the FAM20A gene in blood originated from the abovementioned subject, a step for analyzing the abovementioned measured amount of expression using a gene expression profile that is prepared in advance and correlates with the abovementioned indicator, and a step for estimating the value of the abovementioned indicator in the abovementioned subject on the basis of the abovementioned analysis results.

In one embodiment of the present invention, the amount of expression of the FAM20A gene changes in direct proportion to an indicator of the activity of rheumatoid arthritis and particularly reflects variations in the indicator of activity as described in examples below. As used herein, the term “gene marker” is used substantially as the same meaning as “gene,” is an indicator for evaluating the condition or operation of a certain object and refers to a gene-linked substance that correlates with the amount of expression of a certain gene. By way of example, included are a gene itself, mRNA, which is a transcript, a peptide, which is a translated substance, and a protein, which is the final product of expression of a gene. The term “relative expression frequency” of a gene refers to the normalized amount of expression of the gene and includes the amount of expression on the transcription level of the gene (in the case of a transcript) or the amount of expression on the translation level (in the case of a polypeptide, a protein or the like).

In the present embodiment, the term “rheumatoid arthritis-linked indicator” includes DAS28-CRP, DAS28-ESR, DAS44. EULAR improvement criteria, CDAI, SDAI, ACR and serum CRP concentration, yet the indicator is not particularly limited to those as far as it can be expressed by digitizing and scoring the degree of the condition and progression of rheumatoid arthritis as well as the activity thereof.

DAS (Disease Activity Score) is an evaluation method recommended by DAS (Disease Activity Score), which is an evaluation method recommended by EULAR (European League Against Rheumatism). Moreover, DAS28 is a DAS in which the number of joints to be evaluated has been narrowed down to 28 in order to facilitate its use in daily medical examination and treatment. In DAS28, those 28 joints are measured for four items, i.e., (1) tender joint count, (2) swollen joint count, (3) hourly value of erythrocyte sedimentation rate or CRP (mg/dl) and (4) overall evaluation of condition and then digitized based on a prescribed formula to calculate the absolute value of the activity of disease. When the erythrocyte sedimentation rate is used, it is referred to as DAS28-ESR, and when the CRP value is used, it is referred to as DAS28-CRP. In the present examples, both DAS28-ESR and DAS28-CRP can be used as DAS28. Moreover, DAS44 is used for measuring 44 joints. In the present embodiment, while DAS28-ESR and DAS28-CRP change in proportion to variations in the amounts of expression of the FAM20A gene, the standard values of the amounts of expression (standard amounts of expression) of the FAM20A gene corresponding to specific DAS28-CRP, DAS28-ESR and the like can be altered appropriately in accordance with experimental conditions and, therefore, are not particularly limited to any specific numerical values. In DAS28-ESR, the activity of disease is evaluated high when the value is 5.1 (4.1) or higher, moderate when 3.2 (2.7)<DAS28-ESR=<5.1 (4.1), low when 2.6 (2.3)<DAS28-ESR=<3.2 (2.7) and as remission when it is 2.6 (2.3) or below (the descriptions in the parentheses correspond to DAS28-CRP). As used herein, the term “remission” refers to the state in which the condition of disease has been alleviated temporarily or continuously or has disappeared apparently.

Furthermore, in one embodiment of the present invention, the EULAR improvement criteria can be used when the value of a rheumatoid arthritis-linked indicator is determined in a subject, wherein the basis of the EULAR improvement criteria is DAS28. The therapeutic effect is evaluated at three stages, i.e., good, moderate and no response in combination of two DAS28 values, i.e., a value after therapy as compared with a value before therapy (i.e., a value after administering a drug as compared with a value before administering the drug). In the present embodiment, the therapeutic effect can be evaluated by any of the abovementioned three stages for the subject in proportion to variations in the amount of expression of the FAM20A gene. In the present embodiment, while the EULAR improvement criteria change in proportion to variations in the amount of expression of the FAM20A gene, the standard value of the amount of expression (standard amount of expression) of the FAM20A gene corresponding to specific EULAR improvement criteria can be altered appropriately in accordance with experimental conditions and, therefore, is not particularly limited to any specific numerical value. By way of example, once the amount of change in the expression of the FAM20A gene corresponding to “moderate” is set, the amounts of change in the expression of the FAM20A gene corresponding to “good” and “no response” can also be set appropriately on the basis of the analysis of a gene expression profile.

In one embodiment of the present invention, when the value of a rheumatoid arthritis-linked indicator is determined in a subject, SDAI (Simple Disease Activity Index) can also be used as the indicator. SDAI was developed as an evaluation method for facilitating calculation at a clinical site because the abovementioned calculation formula for DAS was so complicated that it could not easily be calculated at a clinical site. This SDAI is calculated on the basis of tender joint count, swollen joint count, patients' general evaluation and doctors' general evaluation, wherein joints to be evaluated are the same as those in DAS28. In SDAI, the activity of disease is classified and evaluated high when the value is 26 or higher, moderate when 11<SDAI=<26, low when 3.3<SDAI=<11 and as remission when it is 3.3 or below. In the present embodiment, while the value of SDAI changes in proportion to variations in the amount of expression of the FAM20A gene, the standard value of the amount of expression (standard amount of expression) of the FAM20A gene corresponding to a specific value of SDAI can be altered appropriately in accordance with experimental conditions and, therefore, is not particularly limited to any specific numerical value. By way of example, once the amount of expression of the FAM20A gene corresponding to a specific value of SDAI showing the moderate activity of disease is set, the amounts of expression of the FAM20A gene corresponding to specific values showing the other activities such as high activity of disease and low activity of disease can also be set appropriately on the basis of the analysis of a gene expression profile.

Furthermore, in one embodiment of the present invention, when the value of a rheumatoid arthritis-linked indicator is determined in a subject, CDAI (Clinical Disease Activity Index) can also be used as the indicator. CDAI is found by removing CRP from SDAI. In CDAI, the activity of disease is classified and evaluated high when the value is 22 or higher, moderate when 10<CDAI=<22, low when 2.8<CDAI=<10 and as remission when it is 2.8 or below. In the present embodiment, while the value of CDAI changes in proportion to variations in the amount of expression of the FAM20A gene, the standard value of the amount of expression (standard amount of expression) of the FAM20A gene corresponding to a specific value of CDAI can be altered appropriately in accordance with experimental conditions and, therefore, is not particularly limited to any specific numerical value. By way of example, once the amount of expression of the FAM20A gene corresponding to a specific value of CDAI showing the moderate activity of disease is set, the amounts of expression of the FAM20A gene corresponding to specific values showing the other activities such as high activity of disease and low activity of disease can also be set appropriately on the basis of the analysis of a gene expression profile.

In one embodiment of the present invention, when the value of a rheumatoid arthritis-linked indicator is determined in a subject, ACR can also be used as the indicator. ACR refers to ACR improvement criteria, which are classification criteria set by the American College of Rheumatology to show the pathological level of rheumatoid arthritis. The ACR improvement criteria are defined by seven items of an ACR core set. The ACR core set consists of the seven items, i.e., (1) tender joint count, (2) swollen joint count, (3) evaluation of disease by patients, (4) general evaluation of the activity of disease by patients, (5) general evaluation of the activity of disease by doctors, (6) evaluation of physical function by patients, and (7) acute phase reactants. The ACR improvement criteria is, for example, determined as “ACR criteria improved by 20% (ACR20)” in the case that both of the abovementioned (1) and (2) are improved by 20% or more and three out of the remaining five items, i.e., the abovementioned (3) through (7) are improved by 20% or more after treatment as compared with pre-treatment (i.e., after administering a drug as compared with before administering the drug). Similarly, in the case that ACR is improved by 50% and 70%, ACR50 and ACR70 are used as criteria, respectively. As used herein, the simple description of “ACR” indicates the “ACR improvement criteria” unless otherwise specified. In the present embodiment, while the ACR criteria change in proportion to variations in the amount of expression of the FAM20A gene, the standard value of the amount of expression (standard amount of expression) of the FAM20A gene corresponding to specific ACR criteria can be altered appropriately in accordance with experimental conditions and, therefore, is not particularly limited to any specific numerical value. By way of example, once the amount of change in the expression of the FAM20A gene corresponding to ACR50 is set, the amounts in the expression of the FAM20A gene corresponding to ACR20 and ACR70 can also be set appropriately on the basis of the analysis of a gene expression profile.

In one embodiment of the present invention, when the value of a rheumatoid arthritis-linked indicator is determined in a subject, serum CRP concentrations can also be used as the indicator. The normal serum CRP value is 0.3 mg/dl. In the present embodiment, while the serum CRP value changes in proportion to variations in the amount of expression of the FAM20A gene, the standard value of the amount of expression (standard amount of expression) of the FAM20A gene corresponding to a normal serum CRP value can be altered appropriately in accordance with experimental conditions and, therefore, is not particularly limited to any specific numerical value.

Furthermore, in one embodiment of the present invention, the “FAM20A gene” consists of (i) a base sequence according to SEQ ID NO: 1 or (ii) a base sequence that hybridizes with the base sequence according to SEQ ID NO: 1 under stringent conditions, the base sequence being a base sequence encoding a protein having a function equivalent to that of a protein consisting of an amino acid sequence according to SEQ ID NO: 2. It has been known that FAM20A is a secreted protein.

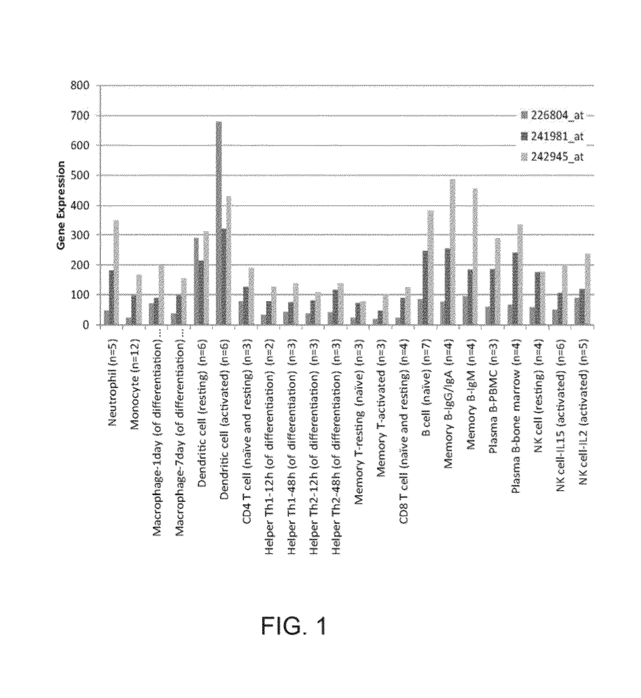

For the purpose of elucidating the biological significance of FAM20A, the present inventors analyzed immune cells contained in blood using a microarray data set by immune cells obtained from NCBI GEO (Gene Expression Omnibus), a publicly available microarray database, in order to find which cell is the source of expression of FAM20A. FIG. 1 shows the result. It was demonstrated that the FAM20A gene was highly expressed in dendritic cells and B cells. It has been known that both dendritic cells, which are antigen-presenting cells, and B cells, which produce antibodies, contribute significantly to the condition of rheumatism. Accordingly, it was biologically assumed that the expression of FAM20A in peripheral blood was highly likely involved in the condition of rheumatoid arthritis.

In one embodiment of the present invention, a method for determining the value of a rheumatoid arthritis-linked indicator in a subject comprises measuring the amount of expression of the FAM20A gene contained in blood collected from the subject. In the determination method according to the present embodiment, the test specimen includes blood collected from a subject, and whole blood, peripheral blood or serum isolated from the blood may also be used as a test specimen. In this case, the method for obtaining serum is not particularly limited, and a wide variety of conventionally known methods may be used (e.g., a method for isolating serum obtained from blood as a specimen for clinical examination). For example, serum may be produced from supernatant obtained by allowing a blood specimen to stand or supernatant obtained by means of centrifugation. Alternatively, a test specimen may be serum in supernatant obtained by placing collected blood in a test tube or the like and then centrifuging it at room temperature under prescribed conditions.

In one embodiment of the present invention, the method for determining the value of a rheumatoid arthritis-linked indicator in a subject facilitates the acquisition of a specimen in that blood originated from a subject can easily be examined as a specimen as compared with a method for collecting and examining synovial fluid and is also advantageous in that a test specimen used in an ordinary clinical examination can be used. Since synovial fluid is collected by an invasive method, patients might be significantly burdened with pain or the like, and there is also some possibility that complications might occur including bleeding and infectious diseases; therefore the examination of synovial fluid is not very recommendable as a routine test method. On the other hand, the abovementioned problems can be solved if the amount of expression of the FAM20A gene is measured using blood as a specimen as shown in the present invention, and the value of a rheumatoid arthritis-linked indicator can easily be determined in a subject. As used herein, the amount of FAM20A refers to the amount of expression of the FAM20A gene unless otherwise specified.

In one embodiment of the present invention, the method for determining the value of a rheumatoid arthritis-linked indicator in a subject uses the FAM20A gene in collected blood as a marker of an indicator for the activity of rheumatoid arthritis. The result of measuring the amount of expression of the FAM20A gene obtained by the method according to the present invention enables to determine an indicator for the activity of rheumatoid arthritis as well as the condition and progression thereof and furthermore enables to diagnose rheumatoid arthritis and determine the therapeutic effect of therapeutic drugs for rheumatoid arthritis.

In one embodiment of the present invention, when the therapeutic effect of a therapeutic drug for rheumatoid arthritis is determined using the method according to the present invention, blood is collected from a subject with rheumatoid arthritis before and after administering the therapeutic drug for rheumatoid arthritis, and then the amount of expression of the FAM20A gene in each of the collected blood is measured before and after administration. Subsequently, the amount of expression of the FAM20A gene in each of the blood is compared with a gene expression profile that is prepared in advance and correlates with a rheumatoid arthritis-linked indicator.

In the present embodiment, as a result of determining the therapeutic effect of a rheumatoid arthritis by the abovementioned method, it has been demonstrated that variations in the expression of the FAM20A gene before and after administering the therapeutic drug is closely related to the evaluation of the actual therapeutic effect. For example, the relationship between the result of measuring the expression of the FAM20A gene and the evaluation by the EULAR improvement criteria can clearly reflects the level of the therapeutic effect (See examples below).

In the present invention. “therapeutic drugs for rheumatoid arthritis” or “drugs for treating rheumatoid arthritis” are not particularly limited and include conventionally well-known therapeutic drugs and all kinds of therapeutic drugs that will be developed in the future. For example, the conventionally well-known therapeutic drugs include biological preparations, non-steroidal anti-inflammatory drugs (anti-inflammatory analgesics), steroidal drugs and immunosuppressants. The biological preparations include chimeric anti-TNF-alpha antibody preparations, soluble TNF receptors, fully human anti-TNF-alpha antibody preparations and anti-IL-6 receptor antibody preparations. The non-steroidal anti-inflammatory drugs include prostaglandin synthesis inhibitors. More specifically, the therapeutic drugs for rheumatoid arthritis according to the present invention include, but are not limited to, methotrexate (MTX), infliximab (IFX), etanercept (ETN), tocilizumab (TCZ), adalimumab (ADA) and abatacept (ABT).

In one embodiment of the present invention, the value of a rheumatoid arthritis-linked indicator can be determined by comparatively analyzing the amount of expression of the FAM20A gene in a subject and a gene expression profile that is prepared in advance from the amount of expression of the FAM20A gene in a sample group of various diseases and health conditions including patients with rheumatoid arthritis, normal individuals and administration or no administration of various therapeutic drugs for rheumatoid arthritis. Alternatively, the value of a rheumatoid arthritis-linked indicator may be determined by comparatively analyzing a gene expression profile of a subject (including normal individuals and patients with rheumatoid arthritis) before administering a therapeutic drug for rheumatoid arthritis and a gene expression profile of the same subject after administering the therapeutic drug for rheumatoid arthritis. In this case, the abovementioned sample group of various diseases and health conditions may be sampled by means of classification on the basis of various elements such as gender, age, type of therapeutic drugs and time after administering a therapeutic drug. As to the gene expression profile that is prepared in advance, the number of accumulated samples is preferably a large as possible in that the accuracy of the determination method according to the present invention increases. Moreover, in the present invention, the constitution may be such that data on such accumulated samples and the gene expression profile that is prepared in advance can be stored in an optional database. In other words, the present invention can provide a database for storing such data and an analyzer for reading and executing such data and programs needed for comparative analysis. Such an analyzer can determine easily and at all times the value of a rheumatoid arthritis-linked indicator in a subject simply by retrieving accumulated data from the database and comparing it with measured data of the subject.

In the present embodiment, the value of a rheumatoid arthritis-linked indicator in a subject can be determined and analyzed by comparing the measured amount of expression of the FAM20A gene with a gene expression profile prepared on the basis of the amount of expression of the FAM20A in various diseases and health conditions, and as to a period after administering a therapeutic drug, any optional period can be set that is clinically significant in evaluating the condition of rheumatoid arthritis disease.

Furthermore, in the present embodiment, it is preferred that the abovementioned gene expression profile show the positive correlation between the amount of expression of the FAM20A gene and the value of a specific rheumatoid arthritis-linked indicator. Accordingly, by way of example, if the amount of expression of the FAM20A gene in blood originated from a subject is higher than the standard amount of expression, the indicator shows the activity of progression higher than the standard value.

In one embodiment of the present invention, as a method for preparing and generating a gene expression profile and analyzing data, any optional analysis means used in the field of microbiology or bioinformatics can be used including cluster analysis and various types of algorisms.

In one embodiment of the present invention, the abovementioned FAM20A gene can also be used as a biomarker for determining the value of a rheumatoid arthritis-linked indicator in a subject. In this case, it is preferred that this biomarker have a base sequence according to SEQ ID NO: 1.

Moreover, in one embodiment of the present invention, an array used for the method for determining the value of a rheumatoid arthritis-linked indicator in a subject is characterized in that each of probes that hybridize with at least part of a base sequence encoding the FAM20A gene under stringent conditions is immobilized at a discrete position on a solid supporter. In the present embodiment, such an array may be substituted by a plate for PCR if necessary, and in this case the constitution is such that the abovementioned probe is placed at a discrete position of wells on the plate.

### (Experiment Method)

The following describes an experiment method and substances used for demonstrating the effect of a gene marker according to one embodiment of the present invention as well as the definition thereof. Although the following experiment method is used in the present embodiment, the similar result can be achieved even when an experiment method other than the method shown below is used.

### (Quantification of Gene)

Furthermore, in the present embodiment, when the gene marker is a gene or a transcript of a gene (mRNA), the amount of expression of the gene can be measured (quantified) by measuring the amount of mRNA by means of various types of microbiological methods including DNA chips, microarray techniques, real-time PCR, Northern blotting techniques, dot blotting techniques and quantitative RT-PCR (quantitative Reverse Transcription-Polymerase Chain Reaction).

The primer used in the quantitative RT-PCR method is not particularly limited as far as it can specifically detect a gene or a gene marker and is preferably an oligonucleotide having 12 to 26 bases. Its base sequence is determined on the basis of sequence information about each human gene. A primer having the determined sequence can be produced using a DNA synthesizer, for example.

On the other hand, when the gene marker is a translated substance (polypeptide) or final product (protein) of a gene, the amount of expression of the gene marker can be measured by means of Western blotting techniques, ELISA techniques, etc. using a polyclonal antibody, a monoclonal antibody and the like specific to the gene marker, yet the method is not particularly limited to those described above and includes various other techniques such as RIA (Radioimmunoassay) and EIA (Enzyme Immunoassay).

The “gene” refers to genetic information represented by base sequences of RNA, DNA, etc. Also included are ortholog genes preserved among species such as human, mice and rats. The gene may be not only one that encodes a protein but one that functions as RNA, DNA or the like as well. The gene generally encodes a protein based on its base sequence but may also be one that encodes a protein whose biological function is equivalent to that of the abovementioned protein (e.g., homologues (homologues, splicing variants), mutants, derivatives). For example, also included is a gene encoding a protein that is slightly different in terms of base sequence from a protein represented by a base sequence according to genetic information, wherein its base sequence hybridizes with a sequence complimentary to the base sequence according to genetic information.

“RNA” includes not only single-stranded RNA but single-stranded RNA having a sequence complimentary thereto and double-stranded RNA constituted therefrom as well. Also included are totalRNA, mRNA and rRNA.

“DNA chips” and “DNA arrays” have a structure in which probe DNA is arranged on a substrate and are used for measuring the expression of multiple genes by means of hybridization. They include not only those used for optically measuring the amount of expression but those that outputs the amount of expression electrically as well. As “DNA chips,” GeneChip® manufactured by Affymetrix, Inc. may be used, for example. As “DNA arrays.” CodeLink Expression Bioarray® manufactured by Amersham Biosciences Corp. may be used. DNA arrays include DNA macroarrays as well as DNA microarrays.

The “amount of expression” includes not only values obtained by directly measuring the amount of expression of a gene but values obtained by conversion by means of prescribed calculation, statistical techniques, etc. as well. Moreover, the “amount of gene expression,” “expression signals.” “gene expression signals.” “values of expression signals,” “values of gene expression signals,” “gene expression data, “expression data,” and the like are interchangeably used to show values that reflect the expression of individual genes.

The “gene expression” refers to the mode of expression of a gene in the living body expressed by the amount of expression of the gene and includes both the case in which it is expressed by the amount of expression of one gene and the case in which it is expressed by a plurality of genes. Additionally, the term “expression” is also used interchangeably to show the mode of expression of a gene in the living body.

### Examples

In the following, the present invention will be described in more detail with reference to examples, yet the present invention is not particularly limited to those examples.

### (Method for Searching for and Selecting Maker Gene)

The following describes a method for searching for and selecting a marker gene according to the present invention.

The present inventors searched for a marker gene using peripheral blood samples of 212 people (402 specimens in total) from patients with rheumatoid arthritis who visited the Department of Rheumatology/Clinical Immunology, Saitama Medical Center, Saitama Medical University, received a written explanation about participation in studies and gave consent. The breakdown of those samples is shown below.

TABLE 1
Breakdown of samples

| Classification | Number of samples |
|---|---|
| MTX0w | 29 |
| MTX4w | 23 |
| IFX0w | 103 |
| IFX2w | 91 |
| IFX22w | 54 |
| IFX54w | 20 |
| ETN0w | 52 |
| ETN2w | 20 |
| TCZ0w | 10 |
| Total | 402 |

RNA was extracted from blood of the test subjects using the PAXgene Blood RNA System (manufactured by QIAGEN, Inc.). We measured the yield of the extracted RNA using NanoDrop1000 (manufactured by Thermo Scientific, Inc.) and made sure that there was no decomposition by Bioanalyzer 2100 (Agilent Technologies, Inc.). Next, we amplified cRNA from 250 ng of RNA by in vitro transcription reaction using Low RNA Input Linear Amp Kit PLUS, One-Color manufactured by Agilent Technologies. Inc. and at the same time fluorescently labeled it (Cy3 labeling). Subsequently, we hybridized the fluorescently labeled cRNA with Whole Human Genome Microarray 4×44k manufactured by Agilent Technologies, Inc. at 65° C. for 17 hours. After washing using Gene Expression Wash Buffer manufactured by Agilent Technologies, Inc., we read fluorescent images by Agilent Scanner (manufactured by Agilent Technologies, Inc.) and then digitized the signal intensity of each spot on the fluorescent images using Feature Extraction, image digitization software manufactured by Agilent Technologies, Inc.

We normalized the digitized data under 75 percentile shift normalization using GeneSpring GX11, microarray analysis software manufactured by Agilent Technologies, Inc. We then calculated a Pearson's correlation coefficient and a p value in the test of no correlation between Log 2 (normalized value) of each probe and DAS28-CRP and DAS28-ESR of a subject to extract a gene that correlates with the activity of disease.

We averaged p values calculated for each probe in the test of no correlation with DAS28-CRP and DAS28-ESR and sorted the data by the average values in ascending order to give a probe list of top 30. Table 2 shows the results.

TABLE 2
Sorted average p values in correlation with
both DAS28-CRP and DAS28-ESR (top 30 probes)

| | Correlation with | | Correlation with | | |
|---|---|---|---|---|---|
| | DAS28-CRP | | DAS28-ESR | | P value |
| Probe D | Cor | P value | Cor | P value | Average |
| A_32_P108254_FAM20A_54757 | 0.608 | 5.08E−42 | 0.616 | 2.07E−43 | 2.64E−42 |
| A_24_P352952_FAM20A_54757 | 0.539 | 1.25E−31 | 0.532 | 8.49E−31 | 4.87E−31 |
| A_23_P330561_C19orf59_199675 | 0.505 | 2.07E−27 | 0.480 | 1.41E−24 | 7.05E−25 |
| A_24_P378202_ARL11_115761 | 0.472 | 1.18E−23 | 0.461 | 1.53E−22 | 8.24E−23 |
| A_23_P21072_NA_NA | 0.465 | 5.73E−23 | 0.461 | 1.47E−22 | 1.02E−22 |
| A_23_P256107_HPSE_10855 | 0.457 | 4.02E−22 | 0.470 | 1.93E−23 | 2.11E−22 |
| A_23_P420873_NR1D1_9572 | −0.455 | 6.04E−22 | −0.478 | 2.16E−24 | 3.03E−22 |
| A_23_P213584_HK3_3101 | 0.464 | 6.70E−23 | 0.454 | 7.90E−22 | 4.28E−22 |
| A_32_P154473_KIF5C_3800 | −0.462 | 1.14E−21 | −0.457 | 3.80E−22 | 7.59E−22 |
| A_23_P51767_CD1C_911 | −0.458 | 3.21E−22 | −0.446 | 4.78E−21 | 2.55E−21 |
| A_23_P351275_UPP1_7378 | 0.447 | 3.44E−21 | 0.449 | 2.40E−21 | 2.92E−21 |
| A_23_P76573_ARL11_115761 | 0.461 | 1.44E−22 | 0.442 | 1.19E−20 | 6.03E−21 |
| A_23_P258088_PACSIN1_29993 | −0.442 | 1.23E−20 | −0.452 | 1.37E−21 | 6.82E−21 |
| A_24_P58054_SLC9A8_23315 | 0.461 | 1.46E−22 | 0.436 | 2.97E−20 | 1.49E−20 |
| A_23_P104471_DUSP13_51207 | 0.435 | 5.05E−20 | 0.453 | 9.24E−22 | 2.57E−20 |
| A_32_P213002_NA_NA | 0.435 | 5.14E−20 | 0.464 | 7.14E−23 | 2.57E−20 |
| A_24_P322741_IL10RB_3588 | 0.438 | 2.70E−20 | 0.431 | 1.24E−19 | 7.54E−20 |
| A_24_P154080_ECE1_1889 | 0.431 | 1.30E−19 | 0.437 | 3.31E−20 | 8.15E−20 |
| A_24_P37962_HK3_3101 | 0.441 | 1.57E−20 | 0.430 | 1.50E−19 | 8.31E−20 |
| A_24_P348265_FCAR_2204 | 0.427 | 2.90E−19 | 0.439 | 2.41E−20 | 1.57E−19 |
| A_23_P253602_BMX_660 | 0.434 | 6.62E−20 | 0.428 | 2.51E−19 | 1.58E−19 |
| A_24_P265523_CR1_1378 | 0.447 | 3.78E−21 | 0.427 | 3.30E−19 | 1.67E−19 |
| A_23_P124559_MXD3_83463 | 0.443 | 9.89E−21 | 0.427 | 3.31E−19 | 1.70E−19 |
| A_23_P158449_SLC37A3_84255 | 0.438 | 2.73E−20 | 0.425 | 4.26E−19 | 2.27E−19 |
| A_23_P169934_RILPL1_353116 | 0.441 | 1.59E−20 | 0.423 | 7.30E−19 | 3.73E−19 |
| A_24_P114255_MBOAT2_129642 | 0.428 | 2.45E−19 | 0.422 | 8.84E−19 | 5.65E−19 |
| A_23_P256868_DCP2_167227 | 0.435 | 5.21E−20 | 0.420 | 1.18E−18 | 6.16E−19 |
| A_23_P148768_F5_2153 | 0.441 | 1.54E−20 | 0.420 | 1.27E−18 | 6.44E−19 |
| A_23_P17655_KCNJ15_3772 | 0.420 | 1.43E−18 | 0.435 | 5.98E−20 | 7.42E−19 |
| A_23_P49376_CETP_1071 | 0.424 | 5.28E−19 | 0.421 | 9.98E−19 | 7.63E−19 |

Two probes on the top were probes for detecting transcription products of FAM20A (Family with sequence similarity 20, member A). In the gene, expression accelerates in peripheral blood as the activity of rheumatoid arthritis disease increases.

### (Analysis of FAM20A Gene)

In the following, the result of analyzing the FAM20A gene according to the present invention will be described.

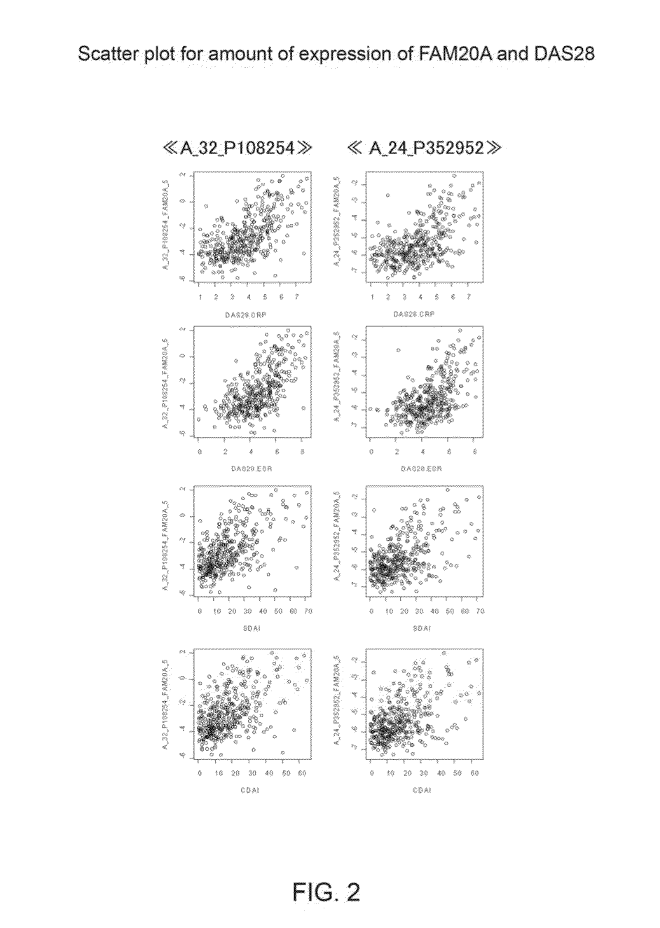

FIG. 2 shows scatter plots between FAM20A Log 2 (normalized values) and DAS28-CRP. DAS28-ESR, SDAI and CDAI. Additionally, Table 3 shows the analysis results of the correlation between FAM20A and clinical examination items other than DAS28.

TABLE 3
Related analysis between values of expression of
FAM20A and RA-linked clinical examination items

| clinical | Correlation with | | Correlation with | | |
|---|---|---|---|---|---|
| examination | A_32_P 108254 | | A_24_P 352952 | | P value |
| items | Cor | P value | Cor | P value | Average |
| CRP | 0.665 | 9.65E−53 | 0.604 | 2.40E−41 | 1.20E−41 |
| DAS28.CRP | 0.608 | 5.08E−42 | 0.539 | 1.25E−31 | 6.27E−32 |
| ESR | 0.664 | 1.87E−52 | 0.534 | 5.00E−31 | 2.50E−31 |
| DAS28.ESR | 0.616 | 2.07E−43 | 0.532 | 8.49E−31 | 4.25E−31 |
| SDAI | 0.568 | 2.14E−35 | 0.522 | 2.57E−29 | 1.29E−29 |
| Albumin | −0.544 | 2.68E−32 | −0.508 | 9.78E−28 | 4.89E−28 |
| D.VAS | 0.518 | 9.05E−29 | 0.472 | 1.61E−23 | 8.05E−24 |
| CDAI | 0.502 | 7.45E−27 | 0.464 | 1.17E−22 | 5.87E−23 |
| TJC48 | 0.452 | 1.16E−21 | 0.424 | 6.30E−19 | 3.16E−19 |
| TJC28 | 0.420 | 1.42E−18 | 0.393 | 2.89E−16 | 1.45E−16 |
| SJC48 | 0.411 | 8.32E−18 | 0.369 | 1.95E−14 | 9.78E−15 |
| Lymphocytes | −0.397 | 8.24E−18 | −0.378 | 2.33E−14 | 1.21E−14 |
| GH | 0.370 | 1.80E−14 | 0.363 | 5.87E−14 | 3.83E−14 |
| P.VAS | 0.376 | 6.34E−15 | 0.361 | 7.73E−14 | 4.18E−14 |
| SJC28 | 0.377 | 5.39E−15 | 0.339 | 2.79E−12 | 1.40E−12 |
| HAQ | 0.351 | 4.29E−13 | 0.338 | 3.26E−12 | 1.84E−12 |
| Platelet count | 0.375 | 8.18E−15 | 0.329 | 1.49E−11 | 7.46E−12 |
| Neutrophiles | 0.333 | 2.68E−11 | 0.340 | 9.86E−12 | 1.83E−11 |
| MMP3 | 0.402 | 2.36E−12 | 0.381 | 3.64E−11 | 1.94E−11 |
| Hemoglobin | −0.295 | 1.70E−09 | −0.283 | 8.06E−09 | 4.88E−09 |
| IgG | 0.341 | 7.12E−09 | 0.248 | 3.53E−05 | 1.76E−05 |
| Total bilirubin | −0.250 | 5.69E−07 | −0.180 | 0.000359 | 1.80E−04 |
| ALT | −0.179 | 0.000311 | −0.199 | 6.02E−05 | 1.85E−04 |
| AST | −0.181 | 2.66E−04 | −0.189 | 0.000141 | 2.04E−04 |
| White blood cell | 0.192 | 1.12E−04 | 0.168 | 0.000728 | 4.20E−04 |
| Total cholesterol | −0.251 | 9.9E−07 | −0.171 | 0.000952 | 4.77E−04 |
| Red blood cell count | −0.179 | 0.000304 | −0.166 | 0.000841 | 5.73E−04 |
| Basophiles | −0.206 | 6.96E−05 | −0.166 | 0.001423 | 7.46E−04 |
| RA test quatitative | 0.208 | 0.003913 | 0.179 | 0.013474 | 8.69E−03 |
| IgA | 0.266 | 8.28E−06 | 0.144 | 0.017615 | 8.81E−03 |
| ALP | 0.190 | 0.00017 | 0.118 | 0.019847 | 1.00E−02 |
| age | 0.115 | 0.020728 | 0.139 | 0.005198 | 1.30E−02 |
| Acidophiles | −0.122 | 0.01943 | −0.133 | 0.010358 | 1.49E−02 |
| IgM | 0.111 | 0.066945 | 0.080 | 0.189462 | 1.28E−01 |
| Mononucleic cells | 0.153 | 0.002775 | 0.049 | 0.344694 | 1.74E−01 |
| Total protein | 0.155 | 0.002037 | 0.046 | 0.366339 | 1.84E−01 |
| Serum creatinine | −0.078 | 0.119193 | −0.040 | 0.420564 | 2.70E−01 |
| gammaGTP | 0.052 | 0.25698 | 0.038 | 0.489683 | 3.73E−01 |
| beta.Dglucan | −0.051 | 0.399982 | −0.044 | 0.468373 | 4.34E−01 |
| BUN | −0.03485 | 0.489196 | −0.03708 | 0.461853 | 0.475525 |
| Anti-dsDNA antibody | −0.04225 | 0.594621 | −0.05651 | 0.476451 | 0.535536 |
| Antinuclear antibody | −0.0559 | 0.474361 | −0.0184 | 0.814013 | 0.644187 |
| LDM | −0.00781 | 0.877473 | 0.012475 | 0.805511 | 0.841492 |

| | | |
|---|---|---|
| Gender (Student T) | 0.408639 | 0.806247 |

This result shows that there is a significant positive con-elation between FAM20A Log 2 (normalized values) and DAS components such as CRP, ESR, TJC, SJC and GH, HAQ (Health Assessment Questionnaire), which is an indicator for evaluating vital functions, and MMP-3, which is believed to increase in rheumatic blood. Accordingly, we believe that by measuring the amount of expression of FAM20A in peripheral blood, we can find the activity of rheumatoid arthritis objectively and comprehensively.

### (Changes in Expression of FAM20A Gene by Administering Drugs)

We examined how the expression of FAM20A changes in the same patient with rheumatoid arthritis before and after drug treatment.

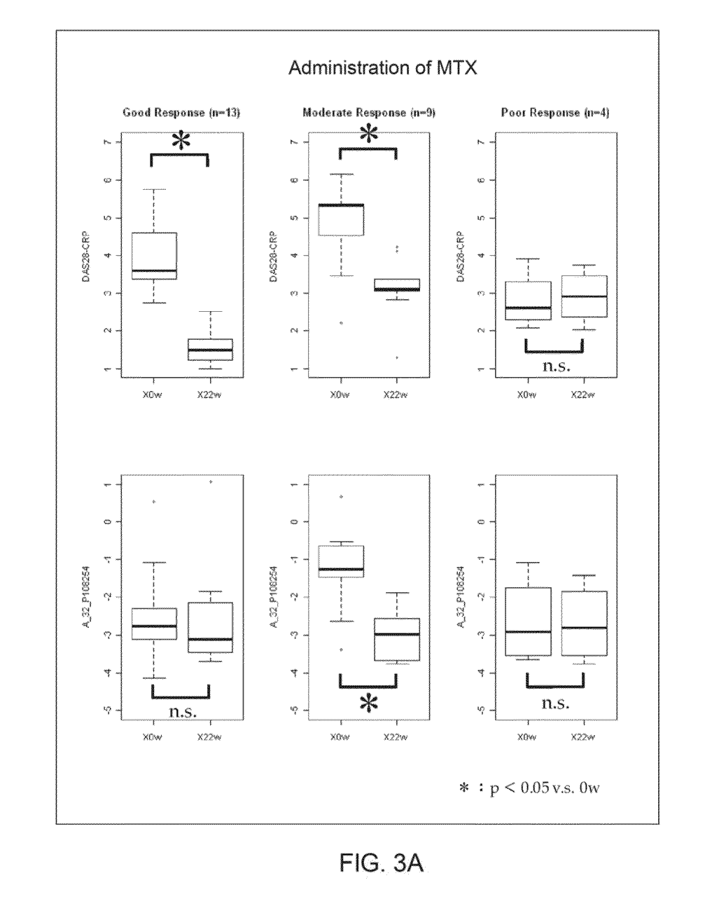

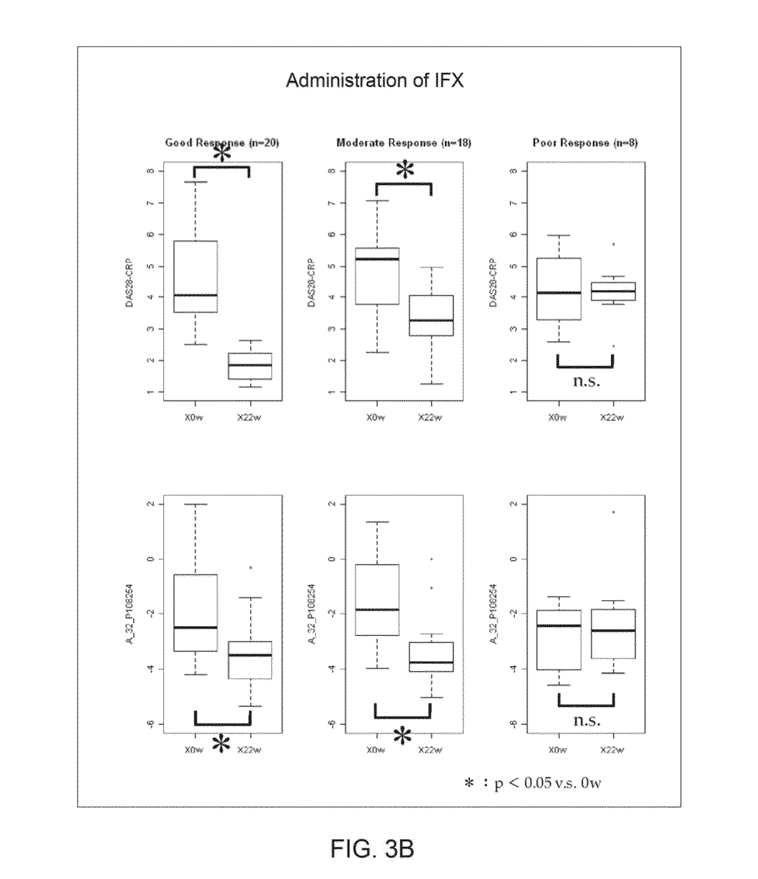

MTX was administered to 26 cases for 22w, and then the result was classified into three groups (good, moderate and poor) on the basis of EULAR improvement criteria, wherein FIG. 3A shows changes in the expression of FAM20A in each group before and after the administration of MTX. The expression of FAM20A declined significantly in 22w as compared with 0w in the moderate response group according to EULAR improvement criteria and also showed the tendency to decline in 22w in the good response group. On the other hand, there was hardly any observable change in the poor response group.

FIG. 3B shows the result after analyzing 46 cases administered with IFX in a manner similar to that described above. The tendency similar to that in the case in which MTX had been administered was observed such that the expression of FAM20A declined significantly in 22w in the good and moderate response groups while there was hardly any change in the poor response group. The abovementioned results suggest that the expression of FAM20A in peripheral blood can be a marker that reflects the effect of a drug for rheumatoid arthritis.

(Comparison with Normal Individuals and Other Diseases)

We compared the amount of expression of FAM20A among patients with rheumatoid arthritis, normal individuals and patients with hepatitis to examine whether or not there was some possibility that FAM20A could be a marker for diagnosing rheumatoid arthritis. Samples obtained from patients with hepatitis were used as a control for inflammatory diseases.

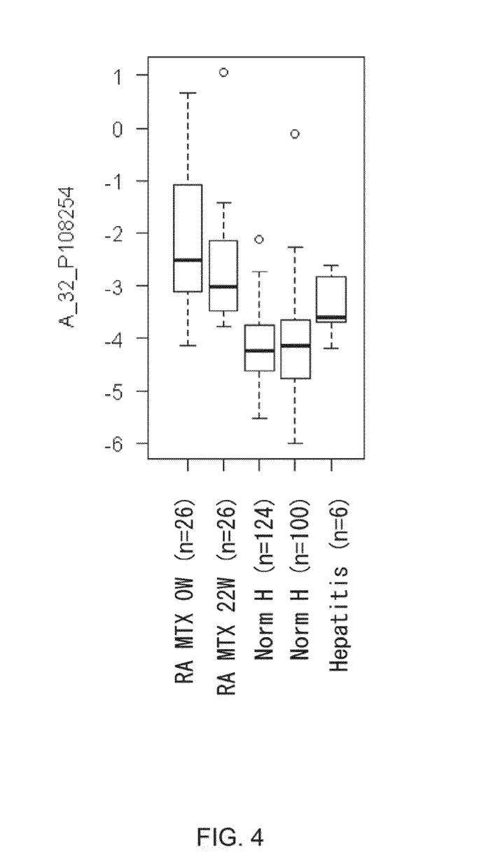

As samples from patients with rheumatoid arthritis, we analyzed MTX 0w and 22w samples in 26 cases administered with MTX as shown in FIG. 3A. As samples from normal individuals, we analyzed peripheral blood samples from 124 people who visited Hishokai Hokkoku Clinic, an incorporated medical institution (Kanazawa City, Ishikawa Prefecture), for the purpose of medical examination as healthy individuals, received a written explanation about participation in studies on the expression of genes in peripheral blood and gave consent (Norm H) as well as peripheral blood samples from 100 people who visited the Public Central Hospital of Matto Ishikawa (Hakusan City, Ishikawa Prefecture), for the purpose of medical examination as healthy individuals, received a written explanation about participation in studies on the expression of genes in peripheral blood and gave consent (Norm M). Additionally, as control samples for inflammatory diseases, we analyzed peripheral blood samples in 6 patients with hepatitis who visited the Department of Gastroenterology. Kanazawa University Hospital, received a written explanation about participation in studies on the expression of genes in peripheral blood and gave consent (Hepatitis). FIG. 4 shows a distribution of FAM20A Log 2 (normalized values) in each sample group.

As a result of the student T test, we found that there was a significant difference (p<0.05) between RA MTX 0w and Norm H, Norm M and Hepatitis as well as between RT MTX 22w and Norm H/Norm M. These results not only show that FAM20A can be used as a marker for evaluating the activity of rheumatoid arthritis but also suggest the possibility that it can be used as a marker for diagnosing rheumatoid arthritis, which is capable of distinguishing patients with rheumatoid arthritis from normal individuals or patients with other inflammatory diseases.

### (Analysis by PCR)

With regard to the “correlation between the expression of FAM20A in peripheral blood and the activity of rheumatoid arthritis in patients with rheumatoid arthritis” and the “difference in the expression of FAM20A in peripheral blood between normal individuals and patients with rheumatoid arthritis,” we validated the results by real-time PCR.

From the abovementioned analysis samples, we picked up 12 cases of normal individuals and 15 cases of patients with rheumatism and performed real-time PCR reaction using a Power SYBR Green RNA-to-CT 1-Step Kit manufactured by Applied Biosystems, Inc. We used 20 ng of RNA in the experiment. As an internal standard gene for correction, we used GAPDH (Glyceraldehyde 3-Phosphate Dehydrogenase). Primers used for detecting FAM20A in the experiment were as follows:

(SEQ ID NO: 3)
Left: CGACACTCCCATGATGAAATC;
and
(SEQ ID NO: 4)
Right: GCTGCAGGTGCAAAAGTGT.

Primers used for detecting GAPDH were as follows:

(SEQ ID NO: 5)
Left: AGCCACATCGCTCAGACAC;
and
(SEQ ID NO: 6)
Right: GCCCAATACGACCAAATCC.

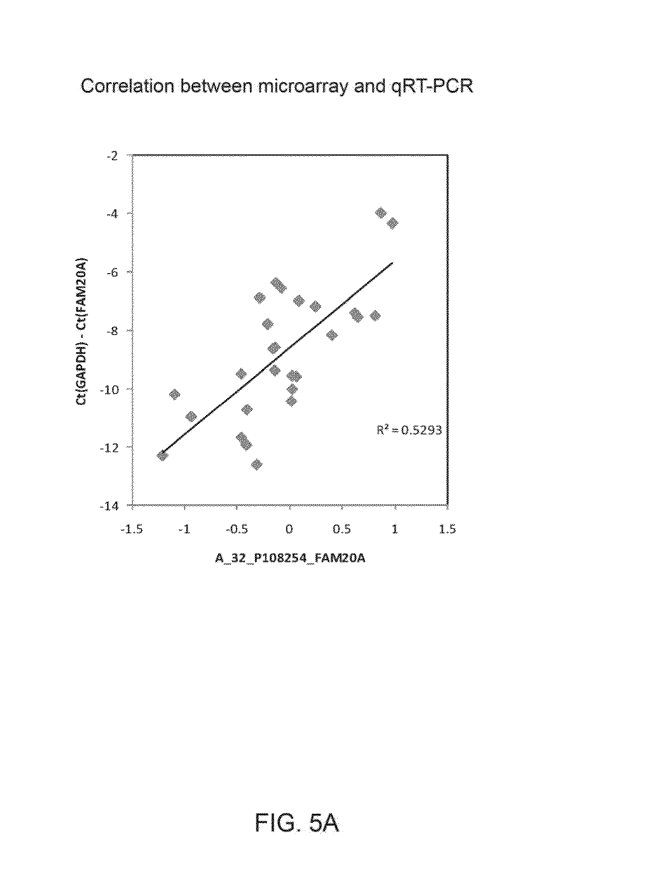

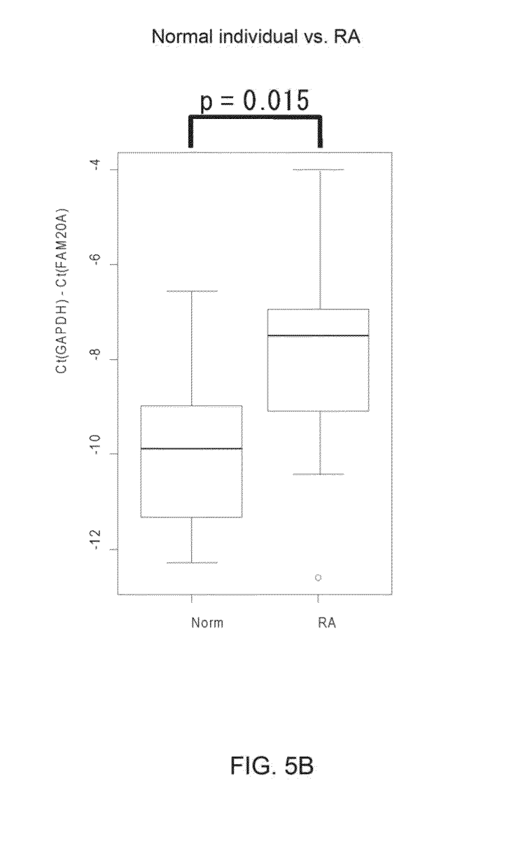

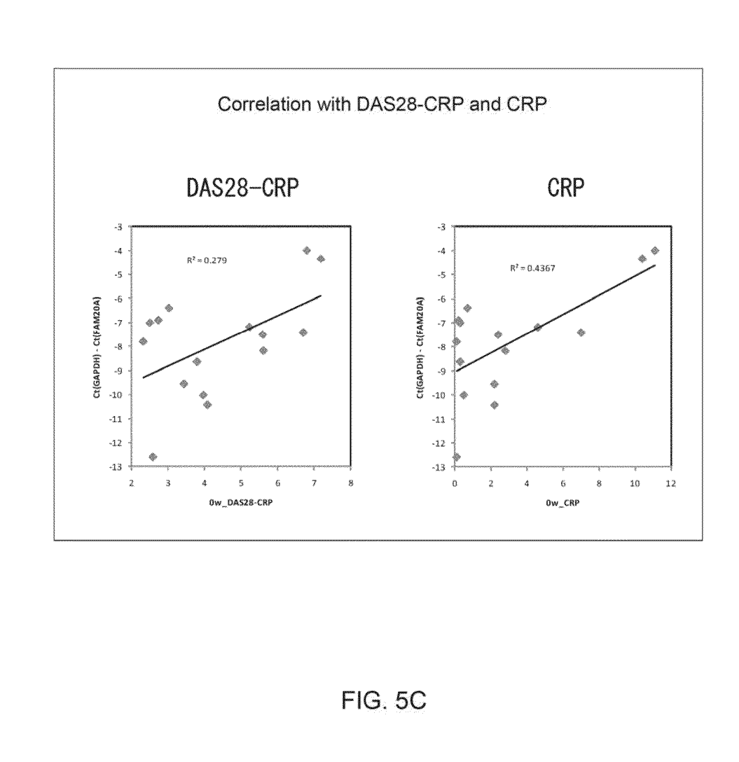

FIGS. 5A-C shows the results.

FIG. 5A shows the correlation between microarray data on FAM20A and PCR cycle number in all of the 27 samples. The correlation of data was very good (R²=0.529). FIG. 5B shows the result of a comparative analysis about the amount of expression of FAM20A quantified by PCR between 12 cases of normal individuals and 15 cases of patients with rheumatoid arthritis. Expression accelerated significantly in patients with rheumatoid arthritis as compared with normal individuals. Furthermore, FIG. 5C analyzes the correlation between the amount of expression of FAM20A quantified by PCR and DAS28-CRP and CRP in the 15 cases of patients with rheumatoid arthritis. As in the case of the microarray data, a significant positive correlation was found between the expression of FAM20A and these clinical indicators.

The abovementioned results clearly show that both the “correlation between the expression of FAM20A in peripheral blood and the activity of rheumatoid arthritis in patients with rheumatoid arthritis” and the “difference in the expression of FAM20A in peripheral blood between normal individuals and patients with rheumatoid arthritis” are reproducible in the real-time PCR as well as in the microarray analysis.

It is to be understood that the present invention can be modified in various manners, i.e., the present invention is not particularly limited to one embodiment as described above but can be modified in various manners without departing from the spirit and scope of the invention.

1. A method for determining a value of a rheumatoid arthritis-linked indicator in a subject, comprising:
measuring an amount of expression of the FAM20A gene in peripheral blood derived from the subject;
analyzing the measured amount of expression using a preliminarily-prepared gene expression profile correlating with the indicator, and
estimating the value of the indicator in the subject on the basis of the analysis results.

2. The method according to claim 1, wherein the indicator is selected from the group consisting of DAS28-CRP, DAS28-ESR, DAS44, EULAR improvement criteria, CDAI, SDAI, ACR and serum CRP concentration.

3. The method according to claim 2, wherein the indicator shows the progression of rheumatoid arthritis or determines a therapeutic effect of therapeutic drugs for rheumatoid arthritis.

4. The method according to claim 1, wherein the analyzing is performed by comparing the measured amount of expression of the FAM20A gene with a gene expression profile that is prepared based on an amount of expression of the FAM20A gene in peripheral blood collected from a patient with rheumatoid arthritis and shows a correlation between an amount of expression of the FAM20A gene and the indicator.

5. The method according to claim 4, wherein the gene expression profile shows a positive correlation, and wherein the indicator shows high activity of progression when the amount of expression of the FAM20A gene in peripheral blood derived from the subject is large.

6. The method according to claim 1, wherein the FAM20A gene consists of (i) a base sequence according to SEQ ID NO: 1 or (ii) a base sequence that hybridizes with the base sequence according to SEQ ID NO: 1 under stringent conditions, the base sequence being a base sequence encoding a protein having a function equivalent to that of a protein consisting of an amino acid sequence according to SEQ ID NO: 2.

7. The method according to claim 1, wherein the subject is suffering from rheumatoid arthritis.

8. The method according to claim 7, wherein the measuring measures amounts of expression of the FAM20A gene derived from peripheral blood before and after administering a therapeutic agent for rheumatoid arthritis to the subject, wherein a therapeutic effect of the therapeutic agent for rheumatoid arthritis is determined by analyzing the amounts of expression of the FAM20A gene in peripheral blood before and after administration using the gene expression profile.

9. A FAM20A marker for determining a value of a rheumatoid arthritis-linked indicator in a subject, comprising a base sequence according to SEQ ID NO: 1.

10. Use of FAM20A comprising a base sequence according to SEQ ID NO: 1 as a biomarker for determining a value of a rheumatoid arthritis-linked indicator in a subject.

11. An array used in the method according to claim 1, wherein a probe that hybridizes with at least part of a base sequence encoding the FAM20A gene under stringent conditions is immobilized at a position on a solid support.


SEQUENCE LISTING (6 sequences)

SEQ ID NO: 1 - DNA, 2267 bp, Homo sapiens
aaaggcgcaagaagcgggcaccccgggaaccccattccctcggctcactcggcgcggagaagcgacgcccgctgactccgagagccccggtgctccgtgcacctggtccccaagttgaggagcgacacccctccacaggggactagcccgcgcggggagcattccggtctcactgaccccggcccacccgcgggactccaggcacctcttctgcccgcaccccgcgacccctcccgggaccccggagacagccggcctgcccccggcgtcccccttggccagcacgccatgccggggctgcgccgggaccgcctactgactctgctactgctgggcgcgctgctctccgccgacctctacttccacctctggccccaagtacagcgccagctgcggcctcgggagcgcccgcgggggtgcccgtgcaccggccgcgcctcctccctggcgcgggactcggccgcagctgcctcggaccccggcacgatcgtgcacaacttttcccgaaccgagccccggactgaaccggctggcggcagccacagcgggtcgagctccaagttgcaggccctcttcgcccacccgctgtacaacgtcccggaggagccgcctctcctgggagccgaggactcgctcctggccagccaggaggcgctgcggtattaccggaggaaggtggcccgctggaacaggcgacacaagatgtacagagagcagatgaaccttacctccctggaccccccactgcagctccgactcgaggccagctgggtccagttccacctgggtattaaccgccatgggctctactcccggtccagccctgttgtcagcaaacttctgcaagacatgaggcactttcccaccatcagtgctgattacagtcaagatgagaaagccttgctgggggcatgtgactgcacccagattgtgaaacccagtggggtccacctcaagctggtgctgaggttctcggatttcgggaaggccatgttcaaacccatgagacagcagcgagatgaagagacaccagtggacttcttctacttcattgactttcagagacacaatgctgagatcgcagctttccatctggacaggattctggacttccgacgggtgccgccaacagtggggaggatagtaaatgtcaccaaggaaatcctagaggtcaccaagaatgaaatcctgcagagtgttttctttgtctctccagcgagcaacgtgtgcttcttcgccaagtgtccatacatgtgcaagacggagtatgctgtctgtggcaaaccacacctgctggagggttccctctctgccttcctgccgtccctcaacctggcccccaggctgtctgtgcccaacccctggatccgctcctacacactggcaggaaaagaggagtgggaggtcaatcccctttactgtgacacagtgaaacagatctacccgtacaacaacagccagcggctcctcaatgtcatcgacatggccatcttcgacttcttgatagggaatatggaccggcaccattatgagatgttcaccaagttcggggatgatgggttccttattcaccttgacaacgccagagggttcggacgacactcccatgatgaaatctccatcctctcgcctctctcccagtgctgcatgataaaaaagaaaacacttttgcacctgcagctgctggcccaagctgactacagactcagcgatgtgatgcgagaatcactgctggaagaccagctcagccctgtcctcactgaaccccacctccttgccctggatcgaaggctccaaaccatcctaaggacagtggaggggtgcatagtggcccatggacagcagagtgtcatagtcgacggcccagtggaacagtcggccccagactctggccaggctaacttgacaagctaagggctggcagagtccagtttcagaaaatacgcctggagccagagcagtcgactcgagtgccgaccctgcgtcctcactcccacctgttactgctgggagtcaagtcagctaggaaggaagcaggacattttctcaaacagcaagtggggcccatggaactgaatctttactccttggtgcaccgcttctgtcgtgcgttgccttgctccgtttttcccaaaaagcactggcttcatcaaggccaccgacgatttcctgagtgcactgggaaatttgggtataggtcaggcttggcagccttgatcccaggagagtactaatggtaacaagtcaaataaaaggacatcaagtgg

SEQ ID NO: 2 - protein, 541 aa, Homo sapiens
MPGLRRDRLLTLLLLGALLSADLYFHLWPQVQRQLRPRERPRGCPCTGRASSLARDSAAAASDPGTIVHNFSRTEPRTEPAGGSHSGSSSKLQALFAHPLYNVPEEPPLLGAEDSLLASQEALRYYRRKVARWNRRHKMYREQMNLTSLDPPLQLRLEASWVQFHLGINRHGLYSRSSPVVSKLLQDMRHFPTISADYSQDEKALLGACDCTQIVKPSGVHLKLVLRFSDFGKAMFKPMRQQRDEETPVDFFYFIDFQRHNAEIAAFHLDRILDFRRVPPTVGRIVNVTKEILEVTKNEILQSVFFVSPASNVCFFAKCPYMCKTEYAVCGKPHLLEGSLSAFLPSLNLAPRLSVPNPWIRSYTLAGKEEWEVNPLYCDTVKQIYPYNNSQRLLNVIDMAIFDFLIGNMDRHHYEMFTKFGDDGFLIHLDNARGFGRHSHDEISILSPLSQCCMIKKKTLLHLQLLAQADYRLSDVMRESLLEDQLSPVLTEPHLLALDRRLQTILRTVEGCIVAHGQQSVIVDGPVEQSAPDSGQANLTS

SEQ ID NO: 3 - DNA, 21 bp, Homo sapiens
cgacactcccatgatgaaatc

SEQ ID NO: 4 - DNA, 19 bp, Homo sapiens
gctgcaggtgcaaaagtgt

SEQ ID NO: 5 - DNA, 19 bp, Homo sapiens
agccacatcgctcagacac

SEQ ID NO: 6 - DNA, 19 bp, Homo sapiens
gcccaatacgaccaaatcc